(19) United States
(12) Patent Application Publication
(10) Pub. No.: US 2008/0071190 A1
(43) Pub. Date: Mar. 20, 2008

(54) Timing of Ovulation Based on Vaginal Ph

(76) Inventors: George Israel Gorodeski, Beachwood, OH (US); Chung Chiun Liu, Cleveland Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/574,279

(22) PCT Filed: Aug. 31, 2005
(86) PCT No.: PCT/US05/30736

§ 371 Date: Oct. 31, 2007

Related U.S. Application Data
(60) Provisional application No. 60/606,033, filed on Aug. 31, 2004.

(51) Int. Cl. A61B 10/00

(52) U.S. Cl. 600/551

(57) ABSTRACT

A method of diagnosing ovulation in a female mammal comprises measuring the pH of the ectocervix of the female mammal and comparing the pH measured of the ectocervix to a reference value.

### RELATED APPLICATION

The present application claims priority from U.S. Provisional Patent Application Ser. No. 60/606,033 filed Aug. 31, 2004, which is herein incorporated by reference in its entirety.

### FIELD OF THE INVENTION

The present invention relates to methods and devices for use in monitoring the ovulation cycle of a female mammal, and particularly relates to a methods and devices for determining when ovulation has occurred in a female mammal.

### BACKGROUND

There is a need to detect and diagnose when female mammals will ovulate, and subsequently whether and when they have ovulated. This information can be of great importance to pinpoint the time for a planned conception. Alternatively, this information can be used as a means of contraception, namely when or when not to have unprotected intercourse.

At present, four methods are commonly available to determine ovulation in females: increases in urine luteinizing hormone (LH); changes in the characteristics of vaginal secretions; increases in plasma progesterone; and ultrasound determination of a decrease in the size of maturing ovarian follicle(s). Additional methods are also present, but they have not been reduced to clinical practice.

The method of measurement of increases in urine LH builds on the physiological increase in plasma and urine LH that precede ovulation by about one to about one and a half days. An over-the-counter kit is available for self-use that requires obtaining a small sample of urine, placing a drop of urine on a platform, and observing a change in color. This so-called “First-Response” would indicate an increase in LH above a baseline predetermined level, and the change in color is defined a predictor of ensuing ovulation. This method has a number of inherent difficulties and problems. It is relatively expensive; one kit costs about $20.00 and can be used for only one menstrual/ovulation cycle. The method has a relatively low accuracy for predicting ovulation, of about 75-85%. An additional difficulty relates to the clinical significance of the increase in plasma/urine LH. While the LH increase triggers ovulation, it precedes ovulation by only about one to about one and a half days. This time-span may be relatively too short for planned intercourse.

A second method for self-determination of ovulation is based on observations of the characteristics of vaginal secretions. In reptoductive-age women the vaginal fluid normally depends on secretions from the epithelial cells of the cervix and vagina, and the characteristics of the fluid change during the menstrual cycle. During most of the cycle (with the exception of the menstrual period), vaginal secretions are scant and relatively thick. However, about 2 to about 3 days prior to ovulation the vaginal secretions become gradually abundant and watery. This change is induced by estradiol, which begins to rise in the plasma already about 6 to about 9 days prior to ovulation, and is the result of an increase in the secretion of fluid from the cervix (the cervical mucus). A number of instruments have been designed for self-exam to determine changes in vaginal secretions. Women would self-insert such an instrument to the vagina, and depending on the device (change of color, change in fluid thickness, etc.) should be able to determine ensuing ovulation. In principle this method is simple and relatively inexpensive, and is used in a number of third-world countries. However, in developed countries, including the US, it had not gained popularity, mainly because of low accuracy. The main difficulty with this method is that the vaginal fluid is a composite of cervical and vaginal secretions, and one may obtain different results of fluid characteristics depending on the placement of the device in the vagina.

This method is nevertheless still used by some physicians to predict ensuing ovulation. In those cases, the patient is examined with the aid of a vaginal speculum; the cervix is visualized for morphology (e.g., color), and for the presence of watery cervical mucus stemming from the cervical os. A drop of the mucus is then used for a Spinbarkeit test (the degree of which the mucus can be stretched), and for pH (pH>7.2 indicates authentic cervical mucus, in contrast to vaginal secretions). As such, this method is cumbersome, requires an exam by an experienced physician, and cannot be used on a routine regular basis.

A third method includes measuring an increase in plasma progesterone. About 6 to about 12 hours after ovulation, progesterone increases significantly in the plasma, and increased plasma progesterone indicates ovulation has occurred. However, measuring an increase in plasma progesterone cannot predict ensuing ovulation. In addition, measuring an increase in plasma progesterone requires blood draw and cannot be used as a self-exam. The test is also relatively expensive, and the results can be obtained about 1 to about 3 days after the blood draw. At present, this test is not used for ovulation determinations but mainly for clinical evaluation of the function of the corpus luteum.

A fourth method to determine ovulation is ultrasound evaluation of decreases in the size of maturing ovarian follicle(s). This test builds on the fact that ovulation involves breakage of the maturing follicle(s), and extrusion of the oocyte into the peritoneal cavity. The decrease in the size of the follicle(s) may correlate with ovulation. Like progesterone determinations, this test can only determine that ovulation had occurred. It involves serial ultrasound tests by experienced personnel in a special setup (e.g., clinic, radiology department, etc.); it is expensive, and therefore it is not used as a routine method to determine ovulation in women.

### SUMMARY OF THE INVENTION

The present invention relates to a method of diagnosing, predetermining, or monitoring the status of the ovulation cycle or fertility phase of an individual mammalian female subject. In the method, the pH of the ectocervix of the female mammal is measured and then compared to a reference value. The reference value can include a predetermined value based on previous pH measurements of the ectocervix. These previous pH measurements of the ectocervix can be charted to determine a threshold value, above which is indicative of ovulation. Optionally, the reference value can comprise pH measurement that was taken along the vaginal wall of the female mammal. This pH measurement can be performed concurrently with the pH measurement of the ectocervix.

The method of the invention has the advantage that it allows with a high degree of accuracy, the determination of an ovulation day and, hence, a fertile phase within a menstrual cycle. When needed for contraception purposes, this leads to a method of prediction of the fertile phase which requires a minimal period of abstinence from unprotected intercourse within any given menstrual cycle.

The present invention also relates to an apparatus for diagnosing ovulation in a female mammal. The apparatus comprises an elongated member that extends along a central axis between a first end and a second end. The second end including an annular side wall sized to contact intimately and circumferentially an outer surface of a ectocervix of a female mammal. The annular side wall including a pH probe for measuring the pH of the ectocervix.

The annular side wall can be radially aligned with the central axis and comprise a pH sensitive material. The second end can further include a tip that comprises a pH sensitive material. The tip can extend from the second end and can be sized to contact intimately an endocervical canal of the female.

In aspect of the invention, the elongated member can be expandable from a first compressed size to a second expanded size and be capable of being received in a sheath when in a compressed size. The sheath can prevent contact of the pH sensitive material of the annular side-wall during insertion of the member into a vagina of the female.

In another aspect of the invention, the elongated member can be a substantially rigid housing. The pH probe of this elongated member can comprise at least one of a pH electrode, thin-film pH sensors, or MEMS pH sensors. The member can further include at least one of a temperature sensor, leuteinizing hormone (LH) sensor, or a pressure sensor.

### BRIEF DESCRIPTION OF THE DRAWINGS

Further features of the present invention will become apparent to those skilled in the art to which the present invention relates from reading the following description of the invention with reference to the accompanying drawings in which:

FIGS. 1(A-B) illustrate schematic cross-sectional views of: (A) the vagina and cervix of a woman; and (B) the vaginal-ectocervical epithelium and endocervical epithelium of the vagina and cervix.

FIGS. 2(A-E) illustrate schematic cross-sectional views of: (A) a vaginal pH device (VPD) in accordance with an aspect of the invention inserted in a vagina of a woman; (B) a lateral pH probe and distal pH probe provided on a vaginal probe of the VPD; (C) the vaginal probe of (B) deployed within the vagina in accordance with an aspect of the invention; (D) the pH detected by the vaginal probe of (C) prior to ovulation; and (E) the pH detected by the vaginal probe of (C) at mid-cycle immediately prior to or during ovulation.

FIG. 3 illustrates a schematic cross-sectional view of a vaginal pH device in accordance with another aspect of the invention.

FIG. 4 illustrates: (A) schema of the experimental system for determinations of luminal pHo (left) and contra-luminal pHo (right) across cultured epithelial cells. Cells are shown as hatched bars. (B) Correlation of pH measurements using the pH electrodes (Elect) and pH paper (pH p) in standard pH solutions. (C) Filled diamonds: stability of pH recordings using blank filter (without cells) in a setup as in (A). Circles in (C) are pH measurements using the pH electrodes (Elect, filled circles) and pH paper (pH p, empty circles) in a blank filter after pH was changed by adding aliquots from 0.1 N NaCl or 0.1 N HCl. (D) Filled circles: pH measurements in a blank filter using the pH electrode (placed in the luminal [L] compartment) after acidification of the contra-luminal solution (filled circles, CL→L). Empty circles: pH measurements in a blank filter using the pH electrode (placed in the contra-luminal [CL] compartment) after acidification of the luminal solution (filled circles, L→CL). Acidification in the cis compartment was induced by adding aliquots of 0.1 N HCl.

FIG. 5 illustrates changes in vaginal and cervical pHo according to the phase of the menstrual cycle. The experiment is described in the text. a-p<0.01 compared to cervical pHo in days 6-9.

FIG. 6 illustrates hECE cells (A), but not human Endocervical Cells (B), acidify constitutively the luminal solution. The experiments are described in the text; shown are determinations of extracellular pH in the luminal (L pHo) and contra-luminal solutions (CL pHo).

FIG. 7 illustrates the effects of estrogen on pHo of the contra-luminal and luminal solutions. hECE cells were plated on filters and shifted to steroid-free medium for one day, and then maintained in the same medium for two additional days in the absence (S.F.M) or presence of 10 nM 17β-estradiol (added to both the luminal and contra-luminal solutions) (+17β-estradiol). For assays, pH electrodes were mounted as described in Methods, and cultures were shifted to basic salt solution in the continued absence or presence of 17β-estradiol. Determinations of pHo were described in the text. Shown are means (±SD) of 3 to 5 repeats per point of pHo determinations 30 min after mounting filters for assays. a-p<0.01 compared to contra-luminal pHo, hECE cells (S.F.M); b-p<0.01 compared to luminal pHo, hECE cells (S.F.M).

FIG. 8 illustrates (A) the specificity of estrogen-decrease in the luminal pHo (L-pHo). The experiments are described in the Legend for FIG. 7. hECE cells were grown in steroid-free medium (SFM) in the absence or presence of one the following hormones (all added at 10 nM to both the luminal and contra-luminal solutions): 17β-estradiol (17βE₂), diethylstilbestrol (DES), estrone (E₁), or testosterone. Shown are means (±SD) of L-pHo determinations in 3 to 5 repeats 30 min after mounting filters for assays. a, b, c-p<0.01 compared to SFM. (B) Dose response effect of 17β-estradiol. The hormone was added at one of the shown concentrations. Shown are means (±SD) of 3 repeats per point of pHo determinations 30 min after mounting filters for assays. The trend of the dose-dependent decrease in mean [L-pHo] vs. [17β-estradiol] was significant (p<0.01).

FIG. 9 illustrates modulation of estrogen-decrease in L-pHo by specific estrogen-receptor modulators (SERMs). The experiments are described in the Legend for FIG. 5. hECE cells were grown in steroid-free medium (S.F.M) in the absence or presence of 10 nM 17β-estradiol, as well as with one of the following SERMs (all added 24 hrs before assays to both the luminal and contra-luminal solutions): 10 μM tamoxifen (TMX), 10 μM ICI-182780 (ICI), or 1 μM progesterone (P₄). Shown are means (±SD) of L-pHo determinations in 3 to 4 repeats per point 30 min after mounting filters for assays. a-p<0.01 compared to control (C), S.F.M group. b-p<0.01, c-p<0.01, and d-p<0.01 compared to S.F.M group.

FIG. 10 illustrates modulation of estrogen-decrease in L-pHo by inhibitors of ATPases. The experiments are described in the Legend for FIG. 7. hECE cells were grown in steroid-free medium (S.F.M) in the absence or presence of 10 nM 17β-estradiol, as well as with one of the following drugs (all added 30 min before assays): Ouabain (1 μM, added to the contra-luminal solution), Omeprazol (100 μM, added to both the luminal and contra-luminal solutions), and Bafilomycin A₁ (1 μM, added to the luminal solution). Shown are means (±SD) of L-pHo determinations in 3 repeats per point 30 min after mounting filters for assays. a-p<0.01 compared to control (C), S.F.M group. b, c, and d-p<0.01 compared to respective treatments in S.F.M group. c-p<0.02 compared to Control, 17β-estradiol group, but p>0.1 compared to omeprazol, 17β-estradiol group. e-p<0.01 compared to b-d; and p<0.01 compared to C, S.F.M group.

FIG. 11 illustrates sidedness of the luminal acidification in hECE cells. The experiments are described in the Legend for FIG. 5. hECE cells were grown in steroid-free medium (S.F.M) plus 10 nM 17β-estradiol, and 30 min before assays Bafilomycin A₁ (1 μM) was added either into the contra-luminal (CL) or into the luminal (L) solution. Determinations of pHo in the contra-luminal (CL-pHo) and luminal solutions (L-pHo) were done as described in Methods. Shown are means (±SD) of 3 to 5 repeats per point 30 min after mounting filters for assays. a, b, c-p<0.01 compared to CL pHo; d-p<0.01 compared to c.

FIG. 12 illustrates that abrogation of the tight junctions modulates the luminal acidification. Filters with hECE Cells were mounted with pH electrodes and shifted to basic salt solution. After 5 min 1.2 mM EGTA plus 1.2 mM CdCl₂ were added to the luminal and contra-luminal solutions from concentrated ×1000 stocks (pH 7.35). Determinations of L-pHo (A) and of CL-pHo (B) were done as described in Methods. The experiment was repeated twice with similar trends.

FIG. 13 illustrates determinations of luminal (l) and contra-luminal (CL) extracellular pH (pHo) in monocultures of hECE cells (M) and in co-cultures of hECE and HCF cells. The experiment was repeated three times with similar trends.

FIGS. 14(A-C) are plots illustrating, respectively, pH levels for 26 woman that were measured along the vaginal wall, the rim of the ectocervix, and at the cervical os. The ph measurements for the women were plotted based on days of the menstrual cycle, i.e., days since onset of menses.

### DETAILED DESCRIPTION

The present invention relates to a method of and apparatus for diagnosing or predetermining the time of ovulation in a female (e.g., human) mammal. The method of the invention is based on the discovery that the estrogen-induced secretion of cervical mucus (such as prior to ovulation) tends to increase vaginal pH in response to increasing levels of estrogen and that this increase in pH can be measured at the cervix of a female mammal to provide a novel method of diagnosing ovulation and the fertile phase of the female mammal. By “fertile phase” it is meant that interval in a female menstrual cycle, spanning the event of ovulation, during which it is most likely that intercourse will result in fertilization, because of normal viability of spermatozoa and ova. Thus, a method is provided wherein substantial increases in the pH at the cervix is diagnostic of impending ovulation and fertility and subsequent decreases in pH after an increased or elevated pH is diagnostic of a decreased susceptibility of fertilization. Additionally, since an increase in estrogen and hence pH at the cervix precedes an increase leuteinizing hormone, the method of the present invention provides an earlier predictor of ensuing ovulation than existing LH methods.

Previous studies in women have shown that under normal conditions vaginal pH ranges from about 5.0 to about 6.0. After menopause, vaginal pH increases to greater than about 7.0, and treatment with estrogen decreases it to premenopausal levels, suggesting that in women estrogen decreases vaginal pH. Utilizing primary cultures of vaginal-ectocervical epithelial cells it was discovered that the cells acidify their luminal surface, but not their subluminal surface, and the effect was potentiated by prior treatment of the cells with physiological concentrations of 17β-estradiol. This newly discovered cellular mechanism explains the general phenomenon of the acidic milieu of the vaginal lumen, and the effect of estrogen.

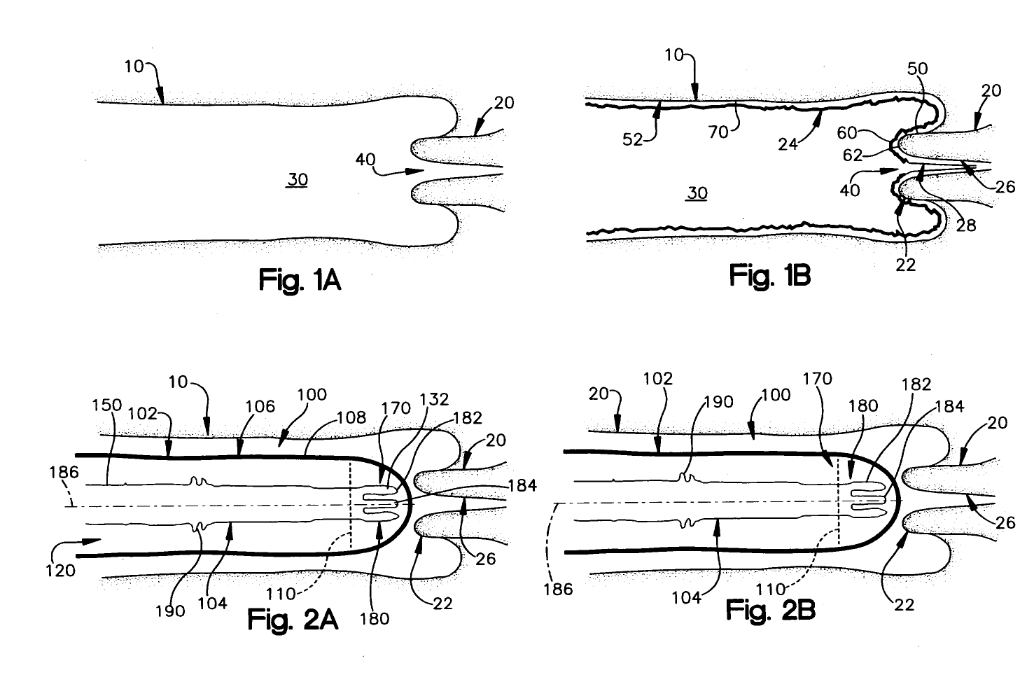

However, in addition to the vaginal epithelial cells, the vaginal pH is also regulated by contributions of cervical secretions. Referring to FIGS. 1A and 1B, which are schematic cross-sectional illustrations of the vagina 10 and the cervix 20, the vagina 10 and outer part 22 of the cervix 20 (“ectocervix 22”) are lined by a stratified squamous epithelium 24, while the inner part of the cervix 26 (“endocervix”) is lined by a simple columnar epithelium 28. As was discussed above, the vaginal-ectocervical epithelial cells 24 actively acidify the vaginal lumen and cause lowering of the pH. The endocervical cells 28 do not participate directly in changing the vaginal pH. Nevertheless, they have an important secondary role that indirectly affects the vaginal pH.

In response to increasing levels of estrogen, the endocervical epithelium 28 becomes permeable (“leaky”) and allows greater diffusion of plasma from the blood into the cervical canal 40 and subsequently into the vaginal lumen 30. Since the cervical plasma is the major component of the cervical mucus, and given that the plasma pH is neutral (e.g., pH of about 7.2 to about 7.4), the estrogen-induced secretion of cervical mucus, such as prior to ovulation, tends to increase vaginal pH. The net effect of estrogen on the vaginal-fluid pH would therefore be the composite of the contributions of estrogen effects on the vaginal-ectocervical epithelium 24 (pH↓) and on the endocervical epithelium 26 (pH↑). Consequently, the measured pH value would greatly depend on the site of sampling, such that single sampling at higher or distal sites 50 (i.e., closer to the cervix 20) would tend to yield higher pH, while sampling at proximal vaginal sites 52 would tend to yield lower pH.

Thus, during most of the menstrual cycle with the exception of the menstrual period, the pH levels close to the cervix 20 will be, for example, about 6.0. However, prior to ovulation, concomitant with increasing plasma estrogen, pH levels close to the cervix 20 will increase, for example, to about 7.2 to about 7.4 due to secretion of the cervical mucus. Accordingly, in the method of the present invention, the pH is measured at a site close to the cervix 20 of the female mammal and compared with a reference value to determine or predict ovulation of the female mammal. For example, a pH measurement and comparison with the reference value that reveals the cervical pH is increasing will indicate impending or ensuing ovulation and fertility of the female. Whereas, a pH measurement and comparison with the reference value that reveals the cervical pH is decreasing from an elevated pH will indicate that ovulation has occurred and susceptibility to fertilization has decreased or ended.

The site 50 close to the cervix 20 where the pH is measured can include at least a portion of the outer surface or circumference of the ectocervix 22 that extends into the vagina 10 or an area of the vagina 10 at least immediately proximate the ectocervix 22. This site should include or be immediately proximate ectocervical epithelium 60 such that during most of the menstrual cycle, with the exception of the menstrual period, the pH of the site 50 will be below that of the endocervical epithelium 28, and immediately prior to and during ovulation, the pH of the site 50 will be higher and comparable to the pH of the endocervical epithelium 50. In an aspect of the method, the site 50 includes a rim portion 62 of the ectocervix 22, which is lined with ectocervical epithelium 60. Although the site 50 can include or be immediately proximate the endocervical epithelium 28, the site should not be limited to just these areas, as the pH of the endocervical epithelium 28 will not substantially change with secretion a cervical mucus.

The pH at the site 50 close to the cervix 20 can be measured using a pH sensor. The pH sensor can include any pH sensor that can be inserted in the vaginal lumen 30 to measure the pH at the site 50 close to the cervix 20. For example, the pH sensor can include at least one pH electrode that is positioned on the distal end of an elongated member that can be inserted in the vagina to the cervix. The pH electrode can be a typical glass pH electrode that includes a silver wire coated with silver chloride, and chloride solution, generally chemically buffered to a pH of 7. Alternatively, the pH sensor can include a thin-film pH sensor, such as a Pd—PdO film sensor, an exemplary discussion of which is found in “A Pd—PdO Film Potentiometric pH Sensor, by Karagounis et al., IEEE Transactions on Biomedical Engineering, Vol. BME-33, No. 2, February 1986 and herein incorporated by reference in its entirety. Yet other pH sensors that can be used include a micro-electromechanical system (MEMS) pH sensor, pH paper, such as pH nitrazine paper, and infrared pH sensors, such as infrared pH sensor disclosed in U.S. Pat. No. 6,542,762 and herein incorporated by reference.

It will be appreciated that pH measurement can comprise single a pH measurement or a plurality or measurements that is taken over a number of days, such as a number of sequential days. Moreover, it will be appreciated that during measurement of the pH at the site 50 close to the cervix 20 care with the pH sensor must be taken to minimize contact of the pH sensor with the mid and lower (i.e., proximal) regions of the vaginal lumen. The mid and lower regions of the vaginal lumen 30 can potentially have a pH that is lower than the cervix, and contact of the pH sensor with the vaginal lumen 30 may adversely affect an accurate measurement of the pH of the ectocervix 22. Accordingly, during the measurement of the pH at the site close to the cervix, the pH sensor can be inserted through a sheath, cannula, and/or vaginal speculum so at to avoid contact with the mid and lower regions of the vaginal lumen.

In an aspect of the invention, the reference value to which the measured pH can be compared can include a reference chart, showing, for example, the female mammal's pH levels with an indication of the time of ovulation on a time coordinate. To form a chart, the pH of the ectocervix can be measured and plotted for a complete ovulation cycle. The chart should include a base-line or threshold pH value (e.g., about 6.0), which is indicative of the period prior to and after increased estrogen levels, and a spike or peak pH value (e.g., about 7.0 to about 7.2), which is indicative of the period of increased estrogen levels and ovulation. Daily determinations of the pH of the ectocervix can be obtained and compared to the chart. When a comparison of the day's test results reveal an increase in pH, for female humans, the fertile period has been reached and ovulation will follow shortly followed by a decrease pH level.

In another aspect of the invention, the reference value can be a predetermined value that is based on measured peak pH values and/or base line pH values at the cervixes of a select population or general population of female mammals. The predetermined value can be a single cut-off value, such a median or mean. The predetermined value can also be a range, for example, where the general population of female mammals is divided equally (or unequally) into groups, or into quadrants, the lowest quadrants being those female mammals with the lowest measured peak or base-line pH values and the highest quadrant being those females with the highest measured peak or base-line pH values. Appropriate ranges and categories can be selected with no more than routine experimentation by those skilled in the art.

Predetermined values of peak and/or base-line pH values, such as for example, mean levels, median levels, or “cut-off” levels, are established by assaying a large sample of individuals in the general population or the select population and using a statistical model, such as a predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference.

In a further aspect of the invention, the reference value can be a pH measurement that is measured at the mid and/or lower portion of the vagina (i.e., the mid and/or lower vaginal wall or lumen) of the female mammal concurrently with the measurement of the pH at the site close to the cervix. The mid and/or lower portion of the vagina is lined with vaginal epithelial cells, which actively acidify the vaginal. The pH value measured at the mid and/or lower portion of the vagina can be compared to the pH value measured close to the cervix. Typically, the pH values measured at both sites will be similar, for example, about 6.0. However, prior to ovulation, concomitant with increasing plasma estrogen, pH levels close to the cervix will increase, for example, to between about 7.2 and about 7.4, while those at the mid and/or lower vagina will either decrease, for example, to about 5.0, or not change. Therefore, with the rise of estrogen which begins about 6 to about 9 days prior to ovulation, there will be a steady increase in the ApH at the site close to the cervix compared to the site at the mid and/or lower vagina.

The ΔpH can be compared with a reference scale or chart that can indicate a range or cut-off point for the ΔpH, which is indicative of ensuing ovulation. Appropriate cut-off points or ranges can be selected with no more than routine experimentation by those skilled in the art. For example, the range or “cut-off” points can be established by assaying a large sample of individuals in the general population or the select population and using a statistical model, such as a predictive value method for selecting a positivity criterion or receiver operator characteristic curve that defines optimum specificity (highest true negative rate) and sensitivity (highest true positive rate).

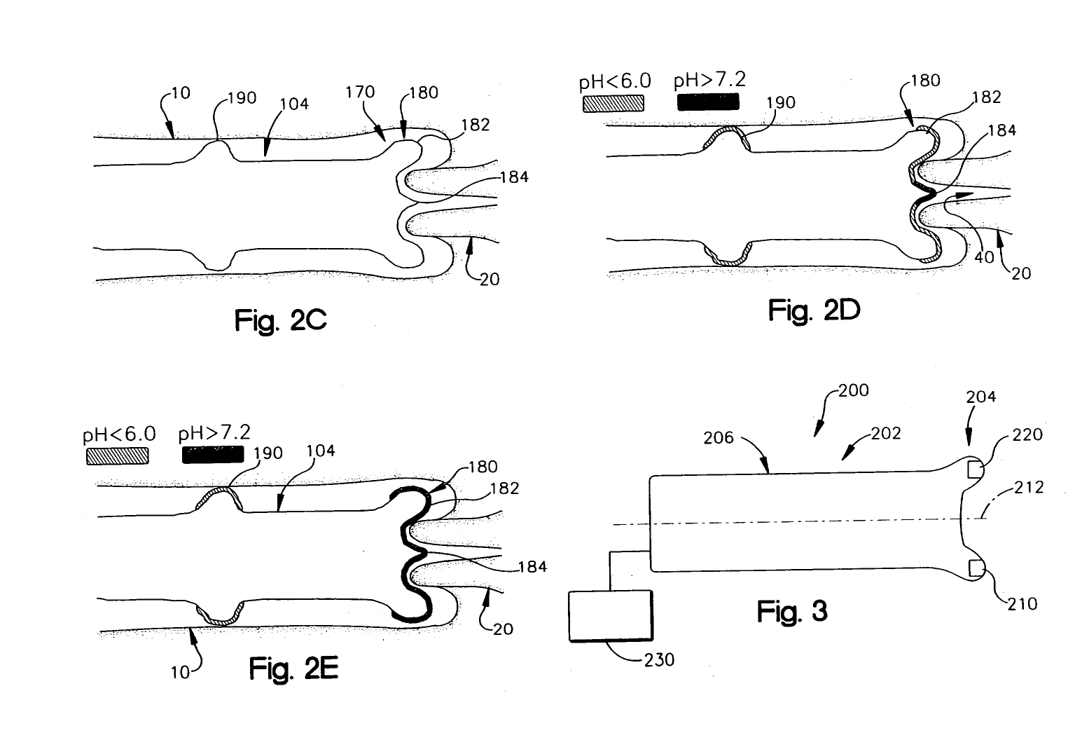

In an aspect of the invention, the pH level at a site 50 close to the cervix 20 and the pH level at a mid-lower region 70 of the vagina 10 can be measured using a vaginal-pH measuring device. The vaginal pH measuring device can be a disposable device that is used for one measurement or a reusable device that can be used to take multiple measurements. An example of a disposable vaginal pH device 100 in accordance with an aspect of the invention is illustrated schematically in FIGS. 2A-2E. Referring to FIG. 2A, the vaginal pH device 100 includes a vaginal sheath 102 and a vaginal probe 104. The sheath 102 comprises a tubular member 106 with a main cylindrical body that extends into a plurality of flexible petal tips 10 disposed at a distal end of the tubular member 106. The tubular member 106 defines a cavity 120 in which the vaginal probe 104 can be provided for insertion within the vagina 10. The sheath 102 prevents the contact of pH sensitive areas of the vaginal probe with vaginal surfaces during its insertion into the vagina 20 of the female subject. The sheath 102 can also facilitate or ease insertion of the vaginal probe 104 in the vagina 10 by maintaining the vaginal probe 104 in a collapsed, compressed, and/or folded state.

The vaginal probe 104 includes a compressible or foldable body 150 that has an anatomic shape, which allows it to be conveniently received in the cavity 120 of the sheath 102 in a compressed or folded state and be conveniently received in the vagina 20 in an unfolded or uncompressed state. The body 150 can be formed from a compressible or foldable material, such as commonly used in the manufacture of tampons. By way of example, this compressible or foldable material can include a cellular or fibrous material, such a cotton web or sponge member.

The body 150 of the vaginal probe 104 in an unfolded or uncompressed state has a generally elongated structure that extends between a first end (not shown) and a second end 170. The second end 170 includes a distal pH probe 180 (or sensor) that is designed to measure the pH at the cervix 20 when the vaginal probe 104 is provided in the vagina 10. The distal pH probe 180 has a funnel or cup-like shape with an annular sidewall 182 and tip 184. The annular sidewall 182 is radially aligned from a central axis 186 and is sized to contact intimately and circumferentially an outer surface of the ectocervix 22. The tip 184 extends axially along the central axis 186 and is sized to contact intimately the endocervical canal 40. The annular side wall 182 and the tip 184 can comprises a pH sensitive material (e.g., phenaphthazine (Nitrazine) pH paper) that will change color to indicate the pH of the cervical mucus. It will be appreciated that the distal pH probe can also include any known pH sensor 180, such as pH electrode, a thin-film pH sensor, or a MEMS pH sensor.

The body 150 of the vaginal probe can also include a lateral pH probe 190 (or sensor) is provided on an outer surface of the body 150 between the first end and the second end 170 of the vaginal probe 104. The lateral pH probe 190 is designed to measure the pH of the mid and/or lower portion of the vagina when the vaginal probe 104 is provided in the vagina 10. The lateral pH probe 190 is annular in configuration and extends about the body 150 of the vaginal probe 104 so that it will contact circumferentially the wall of the vagina 20 at the desired mid and/or lower portion of the vagina 20. The lateral pH probe 190, like the distal pH probe 180, can comprise a pH sensitive material (e.g., phenaphthazine (Nitrazine) pH paper) that can change color to indicate the pH of the vaginal epithelium cells. It will be appreciated that the distal pH probe 180 can also include any known pH sensor, such as pH electrode, a thin-film pH sensor, or a MEMS pH sensor.

In use, the vaginal probe 104 can be compressed or folded and provided in the sheath 102. The vaginal probe 104 and sheath 102 are then inserted into the vagina of the female until the distal end 170 of the sheath 102 reaches the cervix 22. The sheath 102 is disengaged from the vaginal probe 104 by withdrawing the sheath 102 while maintaining the vaginal probe 104 in position with a member (e.g., plunger) (not shown) that can be inserted in a proximal end of the sheath 102.

FIG. 2C illustrates that upon withdrawal of the sheath 102, the vaginal probe 104 will unfold to occupy the vagina 10 and abut the cervix 20. FIG. 2D illustrates that during most of the menstrual cycle (with the exception of the menstrual period) the lateral pH probe 190 will detect a pH of about 6.0, and the distal pH probe 180 will also detect a pH of about 6.0 except at its tip, which corresponds to the area in contact of the endocervical canal 40.

FIG. 2E illustrates that prior to ovulation the lateral pH probe 190 will detect pH levels of about 5.0 to about 6.0, while the distal pH probe 180 will detect pH levels of about 7.2 to about 7.4 in its entirety. The vaginal pH device 100 can also be provided with a scale/table that will indicate numerically the ΔpH between the distal pH probe 180 and the lateral pH probe 190 and a range/cut-off point for the ΔpH to indicate ensuing ovulation.

It will be appreciated by one skilled in the art that other pH measuring devices can be used to measure the pH level at a site 150 close to the cervix 20 and optionally at the mid and/or lower portion of the vagina. FIG. 3 illustrates a pH measuring device (not shown) can be provided with a rod-shaped housing 202 that includes a distal end 204, which is shaped like a mirror image of the ectocervix, and a longitudinally extending body 206. The distal end 204 can have a funnel or cup-like shape with an annular side-wall 210. The annular side-wall 210 is radially aligned about a central axis 212 and is sized to contact intimately and circumferentially the ectocervix.

The rod shape housing 202 can be formed from a substantially rigid, smooth, non-irritating and non-toxic material, such as NYLON, (polyamide) TEFLON (polytetrafluoroethylene), polyethylene, or a silicone, such as a siloxane. The housing can also have an anatomic shape so as to be conveniently received in the vagina of a female mammal. At least one pH sensor 220 can be provided at the distal end 204 of the housing to measure the pH at the ectocervix when the device 200 is inserted in the vagina. The pH sensor 220 can include a pH electrode, a thin-film pH sensor, and/or a MEMS pH sensor.

The pH sensor can be coupled to a processing means 230 that is capable of assimilating, recording, and processing pH measurement data. Based on the pH data, the processing means can determine whether ovulation is impending or ensuing and the fertility of the female. The processing means 230 can further determine whether ovulation has ended and whether the females susceptibility to fertilization has decreased or ended.

The processing means can further include a visual or audio display (not shown). If the processing means determines that a viable egg is present, then a visual display and/or audio component can immediately notify the user that a viable egg is present by signaling. The visual display can display a simple visual indication, such as a combination of colors indicating fertility or infertility (e.g., green for infertile, red for fertile, and yellow for any intermediate stage when conception is less likely but still possible). It will be appreciated that the electronics of a processing means which is capable of assimilating, recording, processing, and displaying pH measurement data, as well as predicting future cycles on the basis of such data can be readily provided by one skilled in the electronics art. The pH measuring device 200 in this example as opposed to previously described pH device 100 may be used for multiple measurements.

It will also be appreciated that additional pH sensors can also be provided on the pH measuring device 200. For example, a plurality of pH sensors (not shown) can optionally be provided along the longitudinally extending body 206 to measure the pH at the mid and/or lower portion of the vagina. These pH sensors like the pH sensor 220 at the distal end 204 of the housing 202 can include pH electrodes, thin-film pH sensors, and/or MEMS pH sensors.

It will further be appreciated that the housing 202 or body 206 of the pH measuring device 200 can include various other sensors with sensing means capable of determining the level of other measurable parameters, for example, other measurable parameters indicative of ovulation (e.g., one or more other parameters that alter as the event of ovulation approaches). For example, the housing 202 or body 206 can further include at least one sensor for measuring the temperature of the vagina and vaginal muscles whenever the housing 202 is inserted in the vagina. One of the methods disclosed in the prior art of determining whether or not ovulation has occurred in a human female is to test for the elevation in body temperature during a woman's fertility cycle. Secretion of progesterone during the latter half of the cycle can raise the body temperature about one-half degree Fahrenheit, the temperature rise coming abruptly at the time of ovulation. During the first half of the menstrual cycle, the temperature fluctuates around 97.6 to 98.0 degree Fahrenheit, then, in a space of 1-2 days, the temperature undergoes a rather steep rise of about 0.9 degree Fahrenheit, to around 98.6 to 99.0 degree Fahrenheit. It remains at this higher level until the next menstrual bleeding. What is important is that, on average, ovulation occurs 1-2 days before the steep rise in temperature.

Keeping in mind this temperature rise, temperature sensors, such as metal-oxide thermistors, can be provided on the housing of the pH measuring device to accurately detect this small change of temperature during the menstrual cycle. Metal-oxide thermistors are formed by mixing together powdered metal oxides, molding them into desired shapes and forming a semiconducting, ceramic-like material whose resistance changes rapidly with temperature. Their response is highly dependent on the oxide mixtures used, and on the manufacturing process. Thermistors used for temperature measurement generally have a negative temperature coefficient. They are narrow range, highly sensitive, nonlinear devices whose resistance decrease with increasing temperature.

Thermistors are offered in numerous configurations. Styles include very small beads, discs ranging from under 0.1 inch to 1 inch or so in diameter, and washers and rods of various dimensions. The thermistor may be coated with epoxy, dipped in glass or otherwise coated, or left unpackaged. The manufacture of precision thermistors begins with careful control and measurement of the slope and stability of each oxide mix. The thermistors are pressed sintered, and metallized, then ground to a precise resistance at a tightly controlled temperature. By proper manufacturing control, precision from ±0.05 to ±0.2 degree centigrade is attainable. For basal body temperature detection, the thermistors can be efficiently calibrated and the corresponding decreased resistance at ovulation can be detected.

An example of another sensor that can be provided on the housing 202 of the pH measuring device is a sensor for measuring leuteinizing hormone (LH) level. It is believed that LH secreted by the anterior pituitary gland causes rapid secretion of the follicular steroid containing a small amount of progesterone. Within a few hours, two events occur, both of which are necessary for ovulation: 1) the capsule of follicle begins to form proteolytic enzymes that cause weakening of the wall, swelling of the entire follicle and the degeneration of stigma and the 2) growth of new blood vessels into the follicle wall, and local hormones are secreted in the follicular tissues causing vasodilation. These two effects contribute to follicle swelling, causing follicle rupture with evagination of the ovum. Approximately two days before ovulation, the rate of secretion of LH by the anterior pituitary gland increases markedly, rising six-to-ten fold and peaking about 18 hours before ovulation. Chemically, the hormones of the anterior pituitary are proteins, and the luteinizing hormone is categorized as proteins. The sensing of this hormone in the detection of the ovulation is therefore advantageous.

A typical LH sensor will transmit and receive an ultrasound of high frequency to detect this hormone. Ultrasound of high frequency can be transmitted at various sites of the housing. On transmission, ultrasound crosses LH protein layers, interacts with vaginal soft tissues, reflects from the surface of soft tissues, crosses again the LH protein layers on its return path, and is collected by an ultrasound receiver. A small size piezoelectric crystal can act as a transmitter and receiver. The presence of LH proteins is determined by the receiving amplitudes of the ultrasound reflected signal. If the reflected signal is weak, it suggests that ultrasound has been absorbed by LH proteins when ultrasound crossed the layers twice on its transmission and reception paths.

A threshold of the ultrasound reflected signal is determined to suggest whether LH hormones are present or not. If the reflected signal is smaller than the threshold value, then it indicates that the LH hormones are present. Caution must be taken to ensure that air is not trapped between the probe and the vaginal soft tissues, as ultrasound is fully reflected from the air interface before ever reaching LH layers.

Still another example of sensor that can be provided on the housing 202 is a pressure sensor for measuring the mucus density at the mouth of the cervix. Ovulation in a woman, who has a normal 28-day female sexual cycle, occurs only 14 days after the onset of menstruation. Shortly before ovulation, the protruding outer wall of the follicle swells rapidly, and a small area in the center of the capsule, called the stigma, protrudes like a nipple. In another half hour or so, fluid begins to ooze from the follicle through the stigma. About two minutes later, as the follicle becomes smaller because of loss of fluid, the stigma ruptures widely, and a more viscous fluid that has occupied the central portion of the follicle is evaginated outward into the abdomen. Once the follicle ruptures, the viscous fluid and mucous collects at the cervical os. The mucous density can be determined by a pressure sensor faced towards the mouth of the cervix.

The pressure sensor can include a thin diaphragm that senses pressure due to a build up of mucous at the cervical os. This pressure is converted into an electrical signal. The larger the mucous density, the more pressure on the diaphragm, (i.e., increased stretching of the diaphragm) thus increased resistance across the diaphragm pressure sensor. The sensor can be attached to the distal of the housing so that it faces the cervical os when the housing is inserted in the vagina. The sensor can be fixed in such a way that it does not realize any pressure coming onto the thin diaphragm from the walls and the mouth of the vagina, except when sufficient mucous builds up due to ovulation. A threshold value is determined for the mucous pressure and is compared with the instantaneous mucous pressure when the data is collected for determining ovulation.

Information about mucous density, basal temperature, and LH level can be transmitted to the processing means 230 along with pH measurement data, if desired. The processing means can then Based on the input data, the processing means can determine whether ovulation is impending or ensuing and the fertility of the female. The processing means 230 can further determine whether ovulation has ended and whether the females susceptibility to fertilization has decreased or ended.

While the form of devices or apparatuses herein described constitutes various aspects of this invention, it is to be understood that the invention is not limited to this precise form of apparatus, and that changes may be made therein without departing from the scope of the invention which is defined in the appended claims. Additionally, while it is expected that the method and device of this invention will find use with humans, the method and device of this invention can also be applied to advantage to female animals whose cervical mucus exhibits hormonal content and changes during estrus similar to those observed in the human female. For example, it is known that in female bovine animals the pre-ovulatory and post-ovulatory changes in reproductive hormones such as progesterone, estrogen, follicle stimulating hormone, and leuteinizing hormone, follow very closely the changes that occur in the human female. Accordingly, the inventive procedure is particularly applicable to cattle and dairy cows for the timing of artificial insemination. Additionally, the procedure can be highly useful for (1) identifying the “silent” periods of estrus; (2) timing of both human and animal embryo transfers; and (3) timing of artificial insemination of wild mammals in captivity.

The following examples are included to demonstrate various aspects of the invention. Those of skill in the art should, in light of the present disclosure, appreciate that many changes can be made in the specific aspects which are disclosed and still obtain a like or similar result without departing from the spirit and scope of the invention.

### EXAMPLES

### Example 1

Estrogen Acidifies Vaginal pH by Upregulation of Proton Secretion via the Apical Membrane of Vaginal-Ectocervical Epithelial Cells.

We proposed that the luminal vaginal pH is determined by net proton secretion into the lumen by vaginal epithelial cells through the coordinated action of ion transport mechanisms located in the apical cell membrane. In this regard vaginal epithelial cells resemble gastric chief cells that regulate net proton secretion into the gastric lumen, as well as other types of cells such as type-A renal intercalated cells, epididymis and vas deferens epithelial cells, macrophages and neutrophils, osteoclasts, cancer metastatic cells, and insects midgut cells. In the present study we initiated experiments in vivo and have begun developing an in-vitro system to test this hypothesis. Our data support the hypothesis, and provide evidence for an estrogen-dependent, bafilomycin-A₁—sensitive proton secreting mechanism in the apical plasma membrane of human vaginal/ectocervical epithelial cells. These data therefore suggest the involvement of estrogen-dependent V-type H⁺-ATPase that regulates acidification of the vaginal canal.

Methods

Vaginal and Cervical pH Determinations In Vivo.

A total of 12 women, ages 20-47 were included in the study. Women were selected from among healthy premenopausal patients presenting for their annual (“well-being”) exam. Included were women with regular menstrual cycles not using hormonal medications and without clinical evidence of vaginal or cervical infections. Based on their last menstrual period women were grouped into three groups according to their Cycle Day as follows: Days 6-9 (n=5), 11-14 (n=3), and 17-24 (n=4). Of the twelve women 8 were African American and 4 were Caucasians. There were no significant differences among the three groups relative to age or gravidity. Prior to their scheduled routine exam all women underwent pelvic examination using nonlubricated vaginal speculum. Using a uterine forceps attached with a strip of pHydrion Paper at its tip (4.5-7.5, Micro Essential Laboratory Inc., Brooklyn N.Y.), the lateral vaginal wall at the level of mid-vagina was gently touched and the Vaginal pH was determined by the change of color of the pH paper strip. The process was then repeated by touching the cervical os, and the Cervical pH was thus determined.

Cell Culture Techniques.

The experiments utilized secondary/tertiary cultures of Human Ectocervical-Vaginal Epithelial Cells (hECE) and of Human Endocervical Cells. Cultures of hECE cells were generated from minces of the ectocervix/vagina. Tissues were collected from a total of 11 premenopausal women ages 37-46; 7 were African American and 4 Caucasians. One woman was from Latino origin. hECE cells were grown and maintained in Dulbecco's modified Eagle's medium (DMEM)/Ham's F12 (3:1) supplemented with nonessential amino acids, adenine (0.2 mM), penicillin (100 U/ml), streptomycin (100 μg/ml), gentamicin (50 ng/ml), L-glutamine (2 mM), insulin (5 μg/ml), hydrocortisone (1 μM), transferrin (5 μg/ml), triiodothyronine (2 nM), epidermal growth factor (0.2 nM) and 8% fetal calf serum, at 37° C. in 91% O₂/9% CO₂ humidified incubator. Culturing techniques and characterization of hECE cells as phenotypically resembling squamous ectocervical/vaginal epithelial cells were described. Cells chosen for experiments were those obtained from tissues reported as HPV negative, and the cultured hECE cells were routinely tested for mycoplasma.

Primary cultures of Human Endocervical Cells were obtained through Clonetics (Walkersville, Md.). Cells chosen for experiments were those obtained from tissues of two women reported as HPV negative, and the cultured Endocervical Cells were tested for mycoplasma. Primary Endocervical cultures were grown and subcultured into second passage using Clonetics proprietary Endocervical Medium (supplied with the cells), and thereafter maintained in hECE culture medium. Cells were characterized as epithelial cells based on typical morphology of a monolayered epithelium; the expression of involucrin; lack of expression of vimentin; and expression of tight junctions (not shown). Confluent endocervical cultures on filters were tested in a diffusion chamber, and were found to generate transepithelial resistance levels of about 35 Ω·cm2, similar to hECE cultures, indicating relatively low resistance cultures. The cells were defined as phenotypically endocervical (in contrast to ectocervical/vaginal) based on expression of apical villi, the abundant expression of cytokeratins 18/19, and minimal expression of cytokeratins 4/5 and 13. Both hECE cells and the Human Endocervical Cells (not shown) expressed functional estrogen receptors.

Co-cultures of hECE cells and human cervical fibroblasts (HCF) were generated by plating irradiated HCF cells on one side of the filter and hECE on the opposing side. Primary cultures of HCF cells were generated from discarded ectocervix/vaginal tissues after the surface epithelium was dissected to generate the hECE cultures. Tissues were immersed in Hank's Balanced Salt Solution (HBSS) plus 2.5% collagenase for 30 min at 37° C. The subepithelial surface was gently scraped with a scalpel; the resulting suspension of cells was incubated for 15 min at 37° C. in a medium composed of Dulbecco's Modified Eagle's Medium and Ham's F-12 (3:1) supplemented with 5% calf serum, L-glutamine (2 mM), penicilllin (100 U/ml), streptomycin (100 μg/ml) and gentamycin (50 ng/ml). Cells were plated in the same medium on plates, and the resulting secondary-tertiary cultures were composed of fibroblasts as determined by morphology, expression of vimentin and lack of expression of involucrin and epithelial-type cytokeratins. Prior to plating on filters HCF cells were irradiated to block further proliferation.

Determinations of Extracellular pH (pHo).

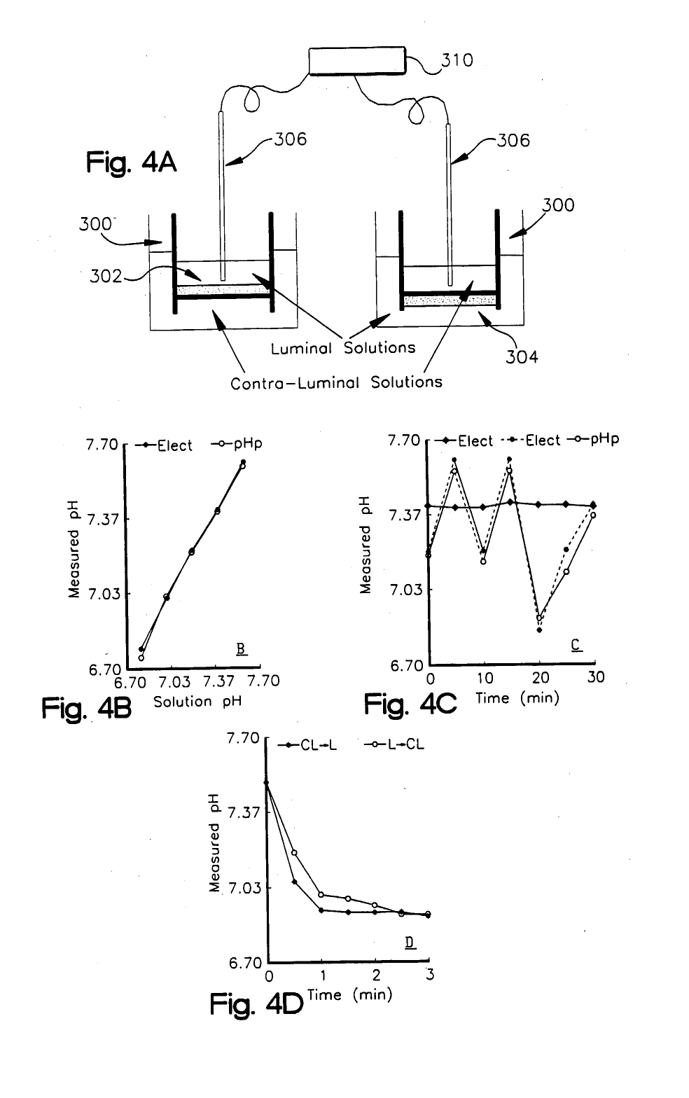

Referring to FIG. 4A, hECE cells or Human Endocervical Cells were plated on Anocell filters (Anocel™-10, Oxon, UK, obtained through Sigma Chemicals, St. Louis, Mo.), which are ceramic-base filters, pore size of 0.02 μm width, 50 μm depth, and surface area of 0.6 cm². Filters 300 and 302 were coated with 4 μg/cm² collagen type IV and incubated at 37° C. overnight. The remaining collagen solution was aspirated and the filter was dried at 37° C. Before plating, filters 300 were rinsed 3 times with Hanks' Balanced Salt Solution. Cells were plated either on the upper surface 302 of one filter 300 (for determinations of luminal pHo) or on the bottom surface 304 of the other filter 300 (for determinations of the contra-luminal pHo) at 3·10⁵ cells/cm². By plating at this relatively high density the cultures became confluent within 12 hours after plating.

Plates containing the filters were placed in a tissue culture incubator (37° C.), and pHo changes in the luminal and contra-luminal solutions were determined using AMANI-1000 microcombination pH electrodes 306 (Harvard Apparatus, Holliston, Mass., obtained through Genomics Solutions, Ann Harbor, Mich.). The AMANI-1000 pH sensor (stored in pH 7.0) is based on metal-metal oxide pH measurements and the pH sensitive layer is immobilized on plastic. The reference electrode employs an Ag/AgCl in 3.4 M KCl reference electrode, and the reference electrode junction is leak-free ensuring no leaks of KCl out of the reference electrode. The design of the pH electrodes 306 enabled their use for the purpose of the present experiments: tip diameter of 1 mm, depth of immersion 1 mm, and response time of less than 5 sec.

Electrodes 306 attached to a stand were held stable with the tip immersed in the solution within the upper compartment of the filter to a depth of about 2 mm avoiding direct contact between the electrode tip and the cultured cells. The electrodes were connected directly to a pH meter 310 (e.g., Accumet pH meter 910) (Fisher Scientific, Suwanee Ga.). After stabilization, the luminal and contraluminal solutions were gently aspirated using glass pipette, and replaced with fresh warmed (37° C.) Basic Salt Solution containing (in mM) NaCl (140), KCl (5), MgCl₂ (1), CaCl₂ (1), glucose (10), and 0.1% bovine serum albumin, pH 7.4. The volumes in the luminal and contraluminal compartments were 150 μl and 500 μl, respectively. Determinations of changes in pHo were made at time 0, and at 5 min intervals thereafter for up to 30 min.

A number of experiments with blank filters (without cells) were done to test the validity of the system, revealing the accuracy (FIG. 4B, 4C) and stability (FIG. 4C) of the pH electrodes. Also shown in FIG. 4D is that acidification of the solution in a cis compartment leads to acidification across the filter in the trans compartment within seconds, indicating that the semi-permeable material of the filter does not impede movement of protons or proton equivalents.

In some experiments pHo determinations were validated using pH paper (Hydrion pH test paper, MSD-2943, Analytical Scientific, Helotes Tex.). Mini strips of the pH papers held by microsurgical pickups were dipped into the luminal solution to a depth of less than 1 mm and changes in pHo were determined by the change of color of the pH paper strip. The results were similar to those using pH electrodes. The data presented in this paper represent pH determinations using the pH electrodes. Similar trends of changes in pHo were obtained if cultures were immersed in 4-(2-hydroxyethyl)-1-piperazineethanesulfonic acid (HEPES)-buffered solution (containing [in mM] NaCl [140], KCl [5], MgCl₂ [1], CaCl₂ [1], glucose [10], and HEPES/Tris [10], pH 7.4) (not shown).

Deprivation of Estrogens.

To remove intracellular estrogens cells were grown in steroid-free medium as described. This procedure involved shifting cells on filters for three days prior to pHo assays to a medium composed of phenol-red-deficient (DMEM)/Ham's F12 or (Sigma Chemicals, St. Louis, Mo.) containing 8% heat inactivated fetal bovine serum that was previously treated with charcoal to remove steroids. Preparation of charcoal-treated serum was described; briefly, dextran-coated charcoal (Sigma Chemicals, St. Louis, Mo.) was dissolved at 8% in 0.15 M NaCl, autoclaved, mixed by stirring, spun and the pellet was resuspended as 1 gm/1.25 ml in H₂O. Fetal bovine serum (Hyclone, Logan, Utah) was mixed with the activated charcoal-dextran at 20:1 (Vol:Vol) and incubated for 45 min at 55° C. At the completion of incubation the mixture was spun twice at 800 g for 20 min and the supernatant (serum) was decanted and collected. For experiments, cells were shifted to steroid-free medium for 3 days; alternatively, cells were shifted to the steroid-free medium for 24 hrs and treated with 17β-estradiol for two additional days.

Cell-Vitality Staining.

Cell-vitality staining was done using the 3-[4,5-dimethylthiazol-2-yl]-2,5-diphenyl tetrazolium bromide (MTT) assay. MTT serves as a substrate for mitochondrial dehydrogenases, and only viable functional mitochondria are capable of cleaving MTT to generate the colored product formazan. MTT solution was prepared by dissolving MTT at a concentration of 5 mg/ml in phosphate-buffered saline (PBS) composed of (mM) NaCl (137), KCl (2.68), Na₂HPO₄ (10), KH₂PO₄ (1.76), pH 7.4, and filtering the solution through Millipore filter. Following treatments/assays, cells on filters were washed with fresh and warm (37° C.) PBS and incubated for 60 min at 37° C. in MTT solution. At the completion of incubation, cells were washed with PBS, and solubilized in isopropanol containing 0.1 M HCl plus 1% Triton X-100. Lysates were mixed by pipetting to dissolve the reduced MTT crystals (during which time the colorless solution turned purple) and were spinned at 10,000 g for 5 min. The solubilized formazan was measured by determining absorption at 570 nm. Background absorbance at 690 nm was subtracted for each sample, and values were normalized to OD₅₇₀ (optic density) of control unperturbed cells.

Statistical Analysis of the Data.

Data are presented as means (±S.D.) and significance of differences among means was estimated by Student's t-test. Trends were calculated using GB-STAT V5.3 (Dynamic Microsystems Inc., 1995, Silver Spring, Md.) and analyzed with ANOVA.

Chemicals and Supplies.

Chemicals and supplies unless specified otherwise, were obtained from Sigma Chemical (St. Louis, Mo.). Stock chemicals and drug solutions were titrated to pH 7.4 or 7.35 prior to cell treatments, and were administered from ×1000 stocks.

Results

pHo Changes In Vivo

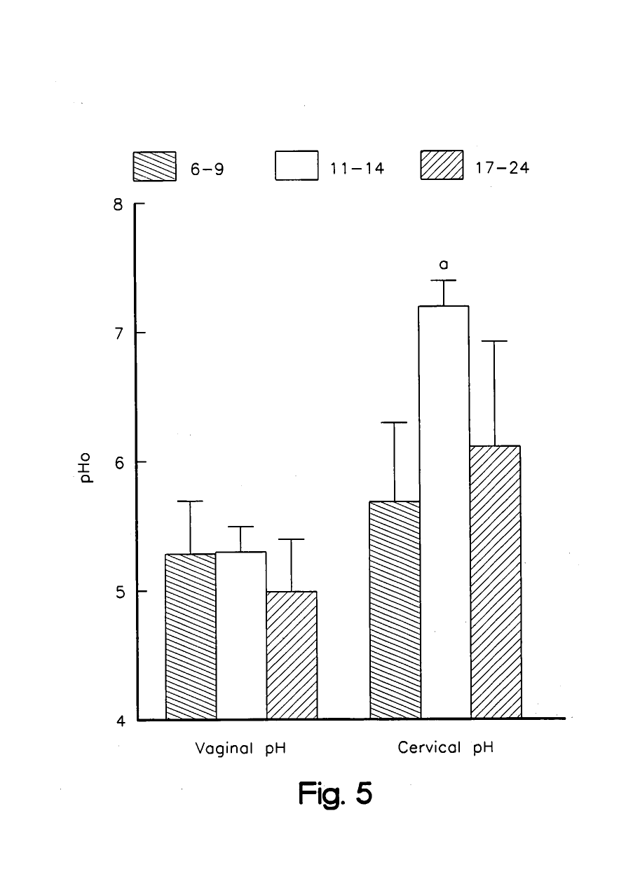

Total of 12 premenopausal women ages 20-47 were included in the study. Measurements of Cervical pHo revealed a mild acidic pHo of 5.7±0.6 in days 6-9 of the menstrual cycle, which increased to 7.2±0.3 during days 11-14 (p<0.01), and remained elevated at 6.1±0.8 during days 17-24 of the menstrual cycle (FIG. 3). In contrast, in the same women Vaginal pH remained low at about 5.1 throughout days 6 to 24 of the menstrual cycle (FIG. 5). The most likely explanation for the changes in Cervical pHo are ovulation-dependent changes in cervical mucus secretion which buffer the acidic pHo at the level of the cervical os. However, in contrast to the changes in Cervical pHo, the menstrual-cycle-related effect was local at the level of the ectocervix, and did not extend distally to the level of the mid-vagina. It is therefore concluded that Vaginal pHo remains acidic throughout the menstrual cycle (FIG. 5).

pHo Changes In Vitro

The levels of Vaginal pHo found in FIG. 5 are significantly lower than the neutral plasma-pH (7.4). One of the explanations is that vaginal-ectocervical epithelial cells secrete acid (protons) actively and selectively away from the epithelium and into the lumen. A similar mechanism was previously reported in a number of different tissues, but until recently little was known about the effect in the vaginal-ectocervical epithelial cells, and the mechanisms involved. To test this hypothesis directly, we generated cultures of human epithelial vaginal-ectocervical and endocervical cells on filters as a model for these experiments. These cultures maintain phenotypic characteristics of the native epithelia (see Methods), and growing cells on filters promotes differentiation and polarization in-vitro. Epithelial-cell polarization is important for the expression of tight junctions and the selective sorting of ion transport mechanisms into apical and basolateral domains of the plasma membrane, that face respectively the luminal and contra-luminal (subluminal) bathing solutions.

Using previously reported methodology, cultures of hECE and Endocervical cells on filters were generated. For most cultures the cells were grown on the upper surface of the filter (facing upwards when the filter is placed horizontally, FIG. 4A, left panel). This established the filter's upper compartment as housing the “luminal” solution, and the compartment in which the filter was bathed as housing the “contra-luminal” solution. In some experiments cells were plated on the bottom part of the filter (by flipping filters vertically). For assays, filters were flipped back to their original position (FIG. 4A, right panel). This established the filter's upper compartment as housing the “contra-luminal” solution, and the compartment in which the filter was bathed as housing the “luminal” solution (FIG. 4A, right panel). Preliminary experiments validated that cultures remain confluent when plated on the bottom part of the filter (not shown).

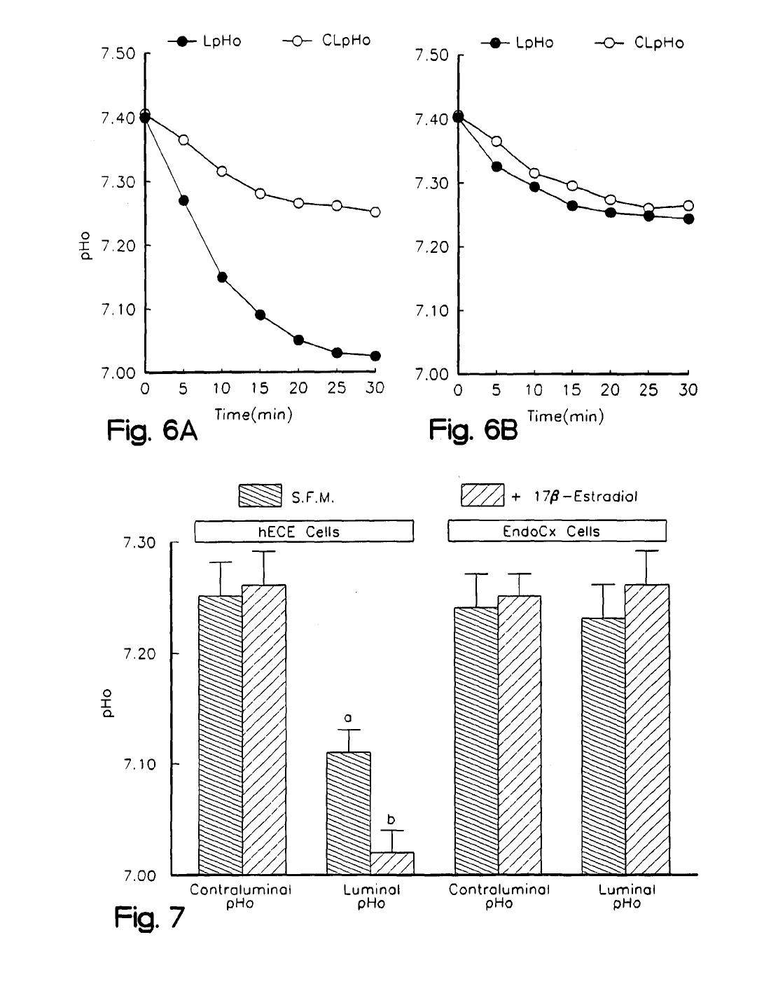

Using this experimental system, it was found that upon shifting hECE cells to basic salt solution, levels of luminal pHo decreased from an initial value of 7.41±0.03 to 7.01±0.04 within 25 min and remained at that level for the duration of the experiment (about 30 min) (FIG. 6A, 5 independent repeats [cells from 3 different women], p<0.01). The mean levels of contra-luminal pHo dropped from 7.41±0.04 to 7.28±0.04 (FIG. 6A, 5 independent repeats [cells from 3 different women], p<0.01). In Endocervical Cells mean levels of luminal pHo and contra-luminal pHo dropped from 7.41±0.04 to only 7.27±0.04 (FIG. 6B, 4 independent repeats [cells from 2 different women], p<0.01).

The most likely explanation for the mild acidification of the contra-luminal pHo in HECE cells and of the luminal and contra-luminal pHo in Endocervical Cells is proton extrusion via the Na⁺/H⁺ exchanger. In contrast, the significant acidification of the luminal pHo in HECE cells suggests constitutive and active net proton extrusion through the apical plasma membrane.

Estrogen Regulates Acidification of Luminal pHo

Previous observations in vivo suggest a role for estrogens in modulating Vaginal pH: estrogen-deficiency, such as after menopause, is associated with alkalinization of vaginal pH and estrogen replacement restores acidic vaginal pH. Based on these observations we hypothesized that estrogen upregulates ion transport mechanisms that mediate the luminal acidification. hECE and Endocervical cells were grown in steroid-free medium to deprive the cells of estrogens, and then treated with 17β-estradiol at the physiological concentration of 10 nM. This low concentration is near maximal for increasing transepithelial, paracellular permeability across cultured human ectocervical-vaginal and endocervical cells.

Incubation of Endocervical Cells in steroid-free medium and treatment with estradiol had no effect on contra-luminal pHo or on luminal pHo, which remained in both cases around 7.25 (FIG. 7). Incubation of hECE cells in steroid-free medium and treatment with estradiol had no effect on contra-luminal pHo, which also remained around 7.25 (FIGS. 7 and 6). In contrast, incubation of hECE cells in steroid-free medium resulted in significant attenuation of the constitutive acidification of the luminal compartment, pHo of 7.11±0.02 (FIG. 5) vs 7.01±0.04 in cells grown in regular culture medium (FIG. 6A, p<0.01). Treatment with estradiol resulted in a significant acidification of the luminal compartment to pHo levels of 7.03±0.02 (FIG. 64, p<0.01). These data indicate that estrogen deprivation attenuates, and treatment with estrogen augments the constitutive net proton extrusion through the apical plasma membrane of hECE cells.

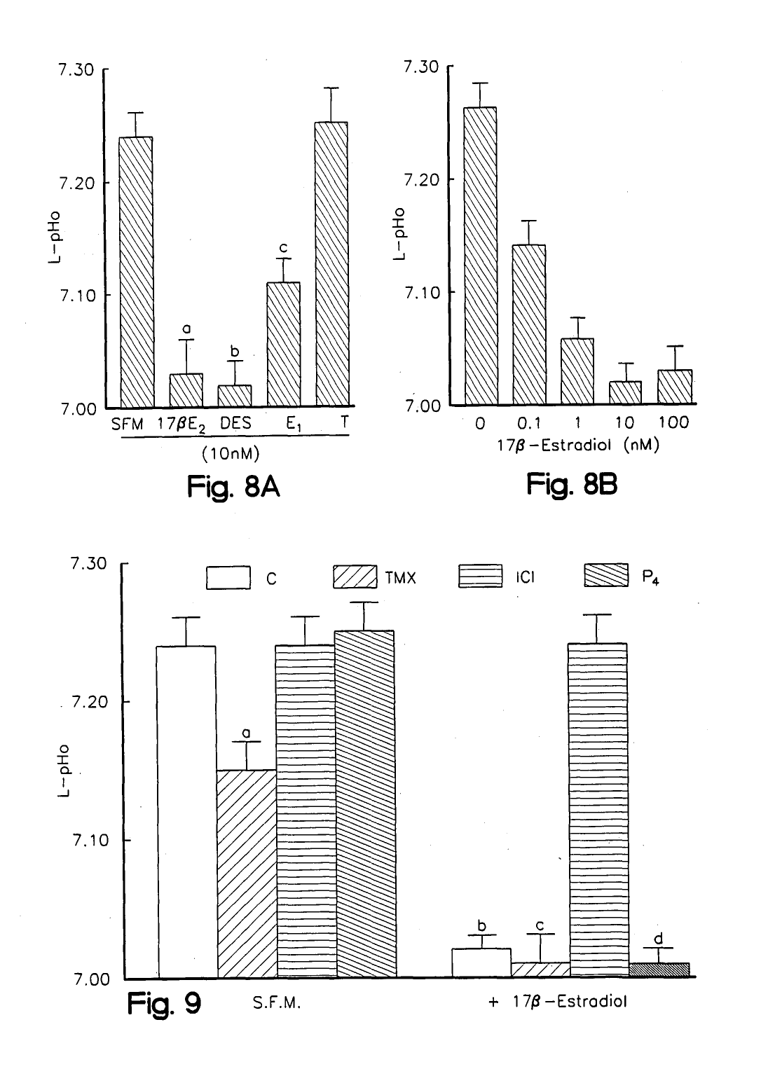

The effect of estrogen was specific, and in hECE cells grown in steroid-free medium only the potent estrogen diethylstilbestrol could mimic 17β-estradiol decrease in luminal pHo. The weak estrogen estrone had only a mild effect, while testosterone had no effect on luminal pHo (FIG. 8A). The effect of 17β-estradiol was dose-dependent: in hECE cells grown in steroid-free medium treatment with the low dose of 0.1 nM of 17β-estradiol already decreased luminal pHo from 7.26±0.03 to 7.13±0.03, and the effect reached near saturation at 1 nM of 17β-estradiol, suggesting an EC₅₀ of 17β-estradiol of about 0.5 nM (FIG. 8B). This level is similar to estrogen effect on paracellular permeability, and is compatible with activation of the classical nuclear estrogen receptor(s) mechanism. Treatment with the higher concentration of 100 nM 17β-estradiol had no additional effect on luminal pHo (FIG. 8B).

SERMs Effects on Estrogen Modulation of Luminal pHo

To determine the degree of which the effect of estrogen could be blocked by specific estrogen receptor modulators (SERMs), hECE cells grown in steroid-free medium were treated with tamoxifen (10 μM), ICI-182780 (10 μM), or with progesterone (1 μM) alone, or in combination with 10 nM 17β-estradiol. Treatment with ICI-182780 or progesterone alone had no effect on luminal pHo (FIG. 9). In contrast, tamoxifen alone decreased luminal pHo from 7.24±0.02 to 7.15±0.02 (FIG. 9, p<0.01). Co-treatment with tamoxifen or with progesterone had no significant effect on the estrogen-induced decrease in luminal pHo (FIG. 9). In contrast, co-treatment with ICI-182780 blocked estrogen-induced decrease in luminal pHo (FIG. 9). Neither tamoxifen ICI-182780 or progesterone had any appreciable effect on contra-luminal pHo (not shown).

Mechanism of the Luminal Acidification

The results in FIGS. 6-9 suggest that hECE cells decrease luminal pHo by active net proton secretion. In other types of cells, several types of H⁺-ATPases have been described which utilize cellular energy by hydrolyzing ATP to effect extrusion of hydrogen ion. These pumps are either electrogenic and require interaction with a parallel ion conductance (e.g., K⁺ channel) to maintain electroneutrality, or involve the countertransport of another cation (e.g. the gastric H⁺/K⁺-ATPase). Three classes of H⁺-ATPases have been described: the E₁-E₂-ATPases (including the Na⁺/K⁺-ATPase [inhibited by ouabain], gastric H⁺/K⁺-ATPase [inhibited by omeprazol], and the Ca²⁺-ATPase); the mitochondrial, chloroplastic and bacterial membranal F₁-F₀-ATPases; and the Vacuolar-type H⁺-ATPases, which are responsible for acidification of extracellular milieus, including the endosomal and lysosomal compartments, and the cell exterior. The latter are also most frequently associated with transepithelial acid secretion.

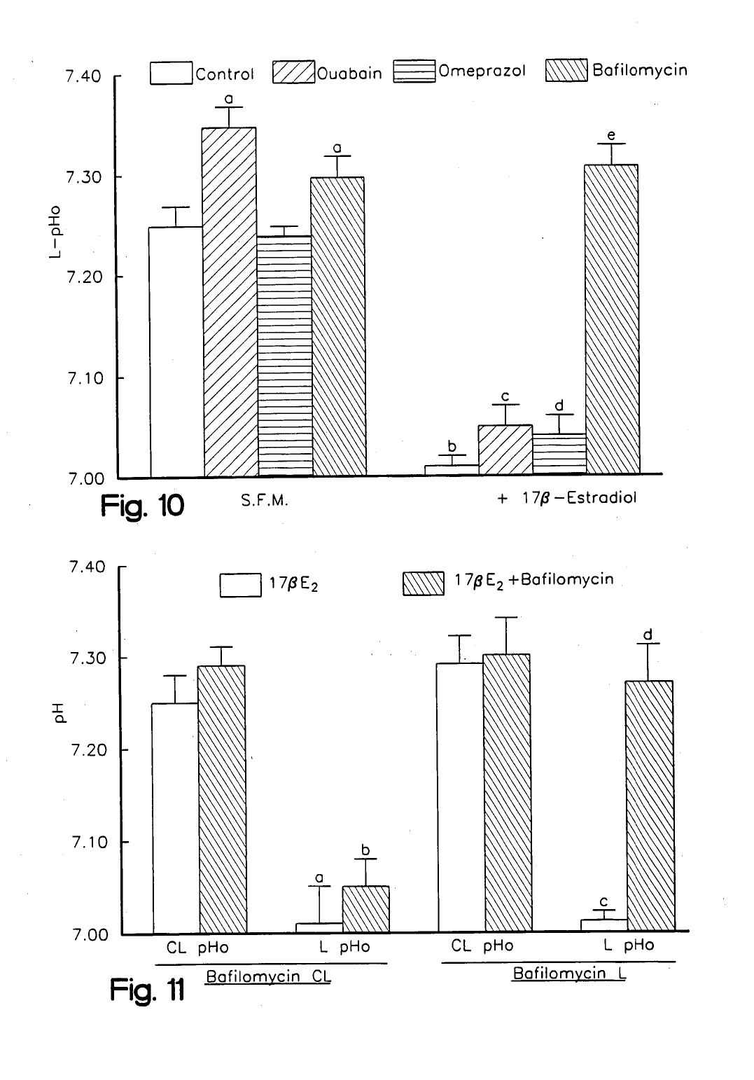

Based on these considerations we hypothesized that the mechanism by which hECE cells decrease luminal pHo involves a Vacuolar-type H⁺-ATPase (V—H⁺-ATPase). This hypothesis was tested by treating hECE cells with a low concentration (1 μM) of bafilomycin A₁, a specific inhibitor of V—H⁺-ATPase. As can be seen in FIG. 10, pre-treatment of hECE cells for 30 min with 1 μM of bafilomycin A₁ blocked the estrogen-induced acidification of the luminal pHo. Moreover, luminal pHo increased to pHo levels measured in the contra-luminal compartment (compare FIG. 10 with FIGS. 6A and 7).

The controls for this experiment were ouabain and omeprazol. Ouabain (1 μM) was used for 30 min prior to the assay, and it was added to the contra-luminal solution to block the Na⁺/K⁺-ATPase that similar to other cells is located in hECE cells in the basolateral membrane (not shown). In cells grown in steroid-free medium pre-treatment with ouabain resulted in mild alkalinization of luminal pHo to levels similar to the contra-luminal pHo (compare FIG. 10 with FIGS. 6A and 7). Pre-treatment with ouabain also attenuated mildly estradiol effect, but luminal pHo still remained significantly more acidic compared to cells not treated with estradiol (FIG. 10). Pre-treatment for 30 min with 100 μM omeprazol (blocker of the gastric type H⁺/K⁺-ATPase) had no effect (FIG. 10). A possible explanation for the results shown in FIG. 9 is that hECE cells express a V—H⁺-ATPase that operates constitutively to acidify the luminal pHo, and that its activity could be upregulated by estrogens. The mild effect of ouabain could be the result of a toxic cellular effect.

Sidedness of the Acidification Mechanism in hECE Cells

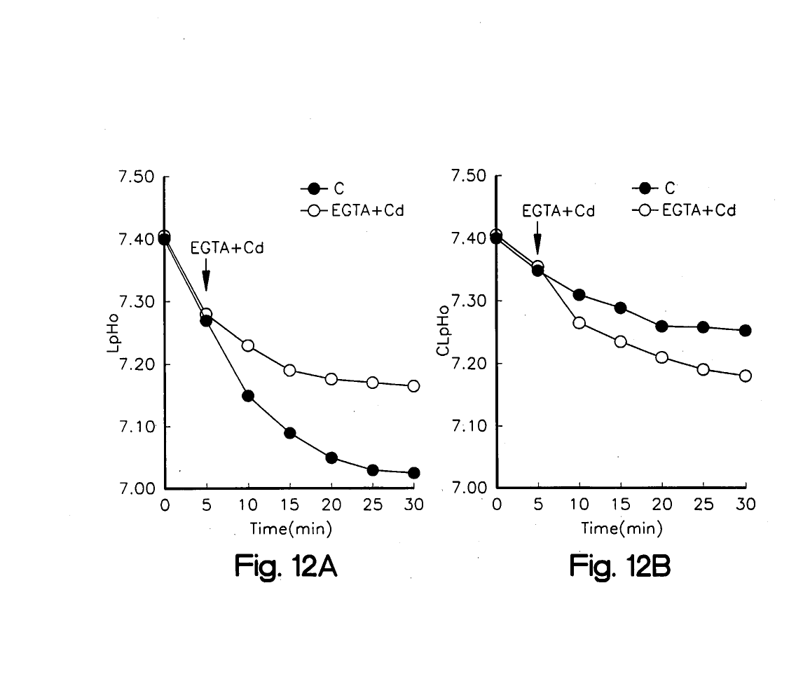

In polarized epithelial cells ion transport mechanisms such as the V—H⁺-ATPase are usually sorted to either apical or basolateral domains of the plasma membrane. This separation is critical for net transepithelial vectorial ion transport. To test more directly the hypothesis that luminal acidification in HECE cells is mediated by apically located proton transport mechanism, bafilomycin A₁ was administered selectively to either the contra-luminal or to the luminal solutions, and effects on pHo were determined in either of these two compartments. In hECE cells grown in steroid-free medium and treated with estradiol pre-treatment for 30 min with 1 μM of bafilomycin, added to the contra-luminal solution, had no effect on the contra-luminal or luminal pHo (FIG. 11, Bafilomycin CL). Pre-treatment for 30 min with 1 μM of bafilomycin A₁ added to the luminal solution also had little effect on the contra-luminal pHo (FIG. 11, Bafilomycin L). However, it significantly blocked the estrogen-decrease in luminal pHo and increased luminal pHo to levels measured in the contra-luminal compartment (FIG. 11). These results indicate that bafilomycin A₁ targets primarily apically-located proton extrusion mechanisms, suggesting that the estrogen-regulated V—H⁺-ATPase is expressed mainly in the apical membrane.

Role of Extracellular Calcium and the Tight Junctions

The involvement of an apically located acidification mechanism suggests a role for the tight junctions in both sorting of the putative V—H⁺-ATPase into the apical membrane, as well as blocking net proton transport through the intercellular space. To test this hypothesis, hECE cultured on filters were treated with 1.2 mM EGTA in order to chelate extracellular calcium to <0.1 mM. It was previously shown that Ca²⁺ is required at extracellular domains of the tight junctions for effective occlusion of the intercellular space, and that lowering extracellular calcium decreases tight junctional resistance within seconds. The Ca²⁺ was replaced with Cd²⁺ that was added to the bathing solutions at the equimolar concentration of 1.2 mM. Cd²⁺ cannot substitute for Ca²⁺ for tight junctional occlusion, and it has minimal effects on permeability across hECE cultures. Lowering Ca²⁺ blocked the luminal acidification (FIG. 12A), and resulted in acidification of the contra-luminal solution (FIG. 12B). Equally interesting is the finding that following treatment with EGTA the luminal pHo and contra-luminal pHo nearly equated at about pH 7.2. These results indicate that in hECE cells the tight junctions are necessary for maintaining luminal acidification.

Co-Culturing with Fibroblasts Augments Proton Secretion

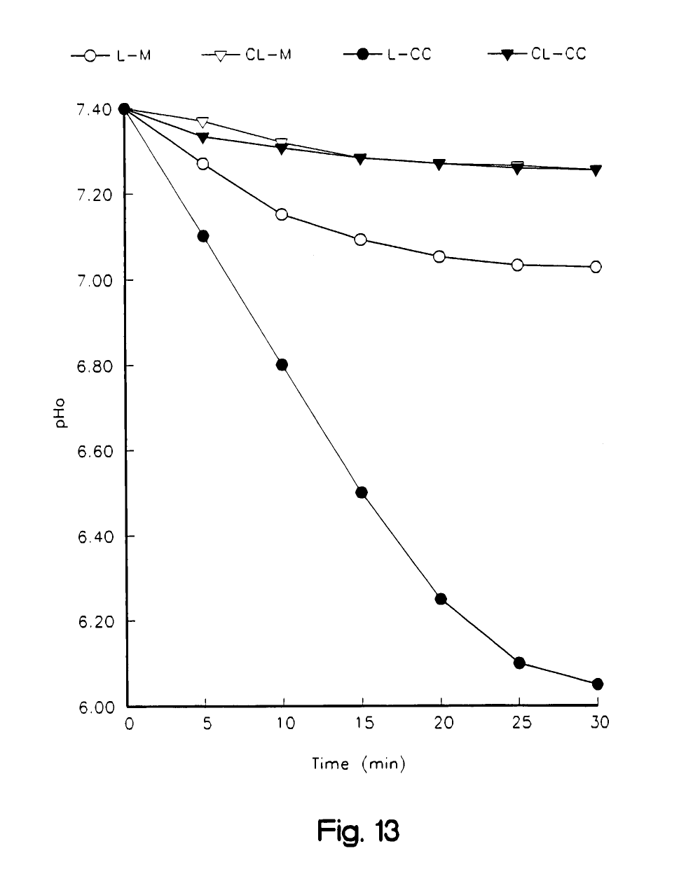

The degreed of luminal acidification in mono-cultures of hECE cells was significant (ΔpHo of about −0.35), but smaller in magnitude compared to the situation in vivo (ΔpHo of about 1.2 to about 2.7). In vivo, epithelial cells rest on a basement membrane that separates them from stromal fibroblasts. To better mimic the in vivo conditions co-cultures of hECE cells and Human Cervical Fibroblasts (HCF) were generated by plating hECE cells and HCF cells on opposite surfaces of the filter (FIG. 4A). Plating irradiated HCF at the relatively low density of 5×10⁴ cells/cm² resulted in non-confluent cultures of HCF, which did not affect per se the electrical resistance across the filter insert (not shown). In addition, in filters plated with only HCF levels of CL-pHo or L-pHo remained at about 7.4 (not shown). Co-plating of HCF improved the attachment of hECE cells to the trans surface of the filter, and resulted in higher transepithelial electrical resistance (about 55 Ω cm², compared to about 35 Ω cm² in monocultures of hECE cells). Co-cultured hECE lowered CL-pHo to pH 7.25 by 30 min, similar to mono-cultures of hECE cells (FIG. 13). In contrast, co-cultured hECE cells lowered L-pHo to 6.05±0.02 (FIG. 13, 4 independent experiments, p<0.01). Thus, the co-cultured hECE acidified the luminal compartment by more than 1 pH unit compared to mono-cultured hECE cells.

The pH Measurements and Treatments Do Not Result in Cell Toxicity

The experimental procedures associated with pH measurements had no effect on cell viability, as determined in terms of the MTT cleavage assay. With the exception of ouabain, all other treatments including incubations in steroid-free medium did not produce significant decreases in MTT cleavage (Table 1). As expected, treatments with ouabain decreased MTT incorporation and staining, and in this context the mild inhibitory effect of ouabain on acidification (FIG. 10) was probably the result of its toxic effect on the cells.

Discussion

The present results show that human vaginal-ectocervical cells express bafilomycin-A₁-sensitive proton secreting mechanism that is located in apical domains of the plasma membrane. The in vivo results showed that vaginal pHo remained acidic throughout the menstrual cycle, and was unrelated to changes in cervical pHo induced by the cervical mucus. We also found that estrogen-deprivation does not abrogate entirely the luminal acidification (FIG. 7). Previous studies in postmenopausal women showed that vaginal pH increases to levels of about 6.5-7.0, which are still lower than plasma pH (7.2-7.4). Collectively these data suggest that the apically-located proton extrusion mechanism acidifies constitutively the luminal fluid regardless of estrogen status. Estrogens up-regulated acidification of the luminal fluid, and a low concentration of 1 nM 17β-estradiol sufficed to exert near maximum effect. This finding suggests that during premenopausal years the acidifying mechanism operates at its near maximum capacity and that cycle-related increases in plasma estradiol do not exert an additional effect on the activity of the proton extrusion mechanism. However, plasma 17β-estradiol below 0.1 nM, such as after menopause, could result in decreased luminal acidification.

At present little is known about which proton extrusion mechanism mediates the luminal acidification in the vaginal-ectocervical cells. The sensitivity to bafilomycin-A₁ and lack of appreciable effects by omeprazol and ouabain suggest involvement of a V—H⁺-ATPase mechanism. Bafilomycin-A₁, like other macrolides (e.g., concanamycins) exerts direct inhibitory effect on the V—H⁺-ATPase by binding to the transmembrane V0 subunit c of the ATPase. Because the effect of bafilomycin-A₁ was most pronounced when the drug was administered to the luminal solution (FIG. 11), it is suggested that in polarized vaginal-ectocervical cells the V—H⁺-ATPase is expressed predominantly in the apical plasma membrane.

Although the V—H⁺-ATPase is probably the main proton extrusion mechanism, other proton transporters could also be involved in the estrogen regulation of pHo in the vaginal-ectocervical cells. HECE express the Na⁺/H⁺ exchanger(s) (not shown) which electroneutrally decrease pHo. Proton secretion via the Na⁺/H⁺ exchanger is driven by the Na⁺_{extracellular→intracellular} gradient, and it is one of the main cellular mechanisms for cell alkalinization. Studies in human breast cancer tissues and in the rat epididymis have shown estrogen regulation of the Na⁺/H⁺ exchanger similar to the effects in hECE cells. In hECE cells the Na⁺/H⁺ exchanger(s) are located predominantly in the basolateral membrane (not shown), and are therefore unlikely to play a role in the acidification of the contra-luminal solution (FIG. 6A). Since neither estrogen-deprivation, nor treatment with 17β-estradiol affected contra-luminal acidification (FIG. 7), it is unlikely that in hECE cells the activity of the Na⁺/H⁺ exchanger(s) is modulated by estrogen.

Na⁺-dependent HCO₃⁻ transporters could also acidify the extracellular milieu, but in contrast to the Na⁺/H⁺ exchanger(s) the main role of HCO₃⁻ transporters is regulation of intracellular pH with minimal contributions to extracellular pH. As such they would probably not play a major role in the acidification of the luminal solution in hECE cells.

Carbonic anhydrase, which is expressed by hECE cells (not shown), could also play a role in the luminal acidification. Members of the carbonic anhydrase family catalyze the reversible reaction CO₂+H₂O⇄HCO₃⁻+H⁵¹, and therefore both produce HCO₃⁻ for transport across membranes and consume HCO₃⁻ that has been transported across membranes. Carbonic anhydrases facilitate trans-cellular CO₂ transport and confer directionality of CO₂ transport across membranes. Carbonic anhydrases also act in concert with membrane-associated ion transport systems such as the Na⁺/H⁺ exchanger. In the rat, carbonic-anhydrase(s) and HCO₃⁻ transporters mediate acidification of the lumen of the vas deferens and epididymis, but these mechanisms have a facilitating role rather than acting as the driving force of proton extrusion.

The mechanism by which estrogen upregulates proton extrusion in hECE cells is not entirely understood. The agonist profile (potency of 17β-estradiol=diethylstilbestrol>estrone>>testosterone), the 17β-estradiol concentration-response profile (EC₅₀≈0.5 nM), and the inhibitory effect by ICI-182780 suggest involvement of the classical estrogen receptor(s) mechanism. The present results differ from effects of estrogens in male rats whereby estrogens inhibit the gastric H⁺/K+-ATPase and hepatic acidification of endocytic vesicles in the liver. In addition, in rat epididymis and vas deferens diethylstilbestrol blocks androgen-dependent expression and activity of the Vacuolar type H⁺-ATPase, which is involved in luminal acidification. Based on these data it appears that the effects of estrogens on V—H⁺-ATPases could be gender related: facilitatory in females, and inhibitory in males.

In hECE cells tamoxifen augmented luminal acidification but to a lesser degree than 17β-estradiol, suggesting partial estrogen agonistic effect. The mechanism of tamoxifen modulation of luminal pHo is unknown. Unlike ICI-182780 tamoxifen did not block the estrogen-increase in acidification. Moreover, tamoxifen effect on luminal pHo differed from its effect on permeability, where tamoxifen attenuates estrogen increase in permeability by blocking estrogen-dependent increase in estrogen-receptor α. These data raise the possibility of non-genomic effect. In fact, it has been suggested that most non-genomic effects of tamoxifen, such as the enhanced drug sensitivity of multidrug-resistant cells, the inhibition of bone resorption and osteoporosis both in vivo and in vitro, and the inhibition of the volume activated chloride channel and calcium channels are the result of tamoxifen inhibition of acidification of cytoplasmic organelles. Using in-vitro assays with isolated organelles and liposomes it was found that tamoxifen increased activity of the V—H⁺-ATPases but it also decreased the membrane potential (Vm) generated by this proton pump. These data raise the possibility of a bimodal role for tamoxifen: enhanced pump activation vs. increased proton permeability. Whether tamoxifen predominant action in hECE involves upregulation of V—H⁺-ATPases activity remains to be determined.

The luminal acidification depended on extracellular calcium. In hECE one of the consequences of lowering extracellular calcium is an acute decrease in tight junctional resistance. Calcium interacts directly with the tight junctions and shifts them into a “closed” state, while chelation of extracellular calcium confers an “open” state. Lowering extracellular calcium blocked luminal acidification and resulted in augmented contra-luminal acidification, suggesting equilibration of the pH across the cultures hECE epithelia. A possible explanation could be increased paracellular permeability to protons; subsequently protons or proton equivalents could move via the intercellular pathway down their electrochemical gradient from the luminal solution (higher H⁺ concentration) to the contra-luminal solution (lower H⁺ concentration). Another explanation is the loss of a restrictive mechanism (tight junctions) for the lateral movement of plasma membrane proteins. According to this speculation abrogation of the tight junctions would allow re-sorting of the proton extrusion mechanism from the apical to the basolateral membrane, resulting in equal acidification of the luminal and contra-luminal solutions.

In conclusion, the present data provide a novel mechanistic explanation for the vaginal luminal acidic pH. Based on these data we advance the hypothesis that vaginal-ectocervical cells acidify the luminal canal by a mechanism of active proton secretion, possibly a V-type H⁺-ATPase that is located predominantly in the apical membrane. We also propose that active net proton secretion occurs constitutively throughout woman's life, but the degree of acidification is estrogen dependent. The mechanism of estrogen effect is at present unclear, although our data suggest involvement of the classical estrogen receptor(s) mechanism.

TABLE 1
Effects of pHo measurements, steroids deprivations and of
the various treatment protocols on hECE cell vitality.

| pHo | Baseline | 30 min |
|---|---|---|
| Basic Salt Solution | 100% | 96 ± 4 |
| | (OD₅₇₀ 0.52 ± 0.02) | |
| Steroid-Free Medium | 95 ± 3 | 96 ± 3 |
| +tamoxifen (10 μM) | 93 ± 4 | 95 ± 4 |
| +ICI 182,780 (10 μM) | 95 ± 8 | 95 ± 3 |
| +Progesterone (1 μM) | 95 ± 5 | 95 ± 4 |
| +Ouabain (1 μM) | 71 ± 8^{a,b} | 69 ± 9^{b} |
| +Omeprazol (100 μM) | 92 ± 5 | 95 ± 4 |
| +Bafilomycin A₁ (1 μM) | 94 ± 3 | 95 ± 4 |
| +CdCl₂ (1.2 mM) | 94 ± 2 | 95 ± 9 |
| Steroid-Free Medium + 10 nM | 96 ± 3 | 94 ± 4 |
| 17β-Estradiol | | |
| +tamoxifen (10 μM) | 95 ± 3 | 96 ± 4 |
| +ICI 182,780 (10 μM) | 89 ± 9 | 93 ± 6 |
| +Progesterone (1 μM) | 91 ± 6 | 95 ± 4 |
| +Ouabain (1 μM) | 62 ± 4^{a,b} | 74 ± 9^{b} |
| +Omeprazol (100 μM) | 91 ± 6 | 95 ± 4 |
| +Bafilomycin A₁ (1 μM) | 93 ± 7 | 92 ± 5 |
| +CdCl₂ (1.2 mM) | 95 ± 2 | 95 ± 3 |

Footnote for Table 1: Means (±SD, 3 to 4 repeats per point) of changes in cell vitality, expressed in terms of MTT accumulation. The experiments are described in the text, and changes in MTT staining were normalized to OD₅₇₀ of control unperturbed cells maintained in regular medium. a-p<0.01-0.05 compared to the Regular Medium group. b-p<0.01 compared to the other conditions in the Baseline or 30 min pHo measurements categories.

### Example 2

Regulation of Vaginal pH in Women

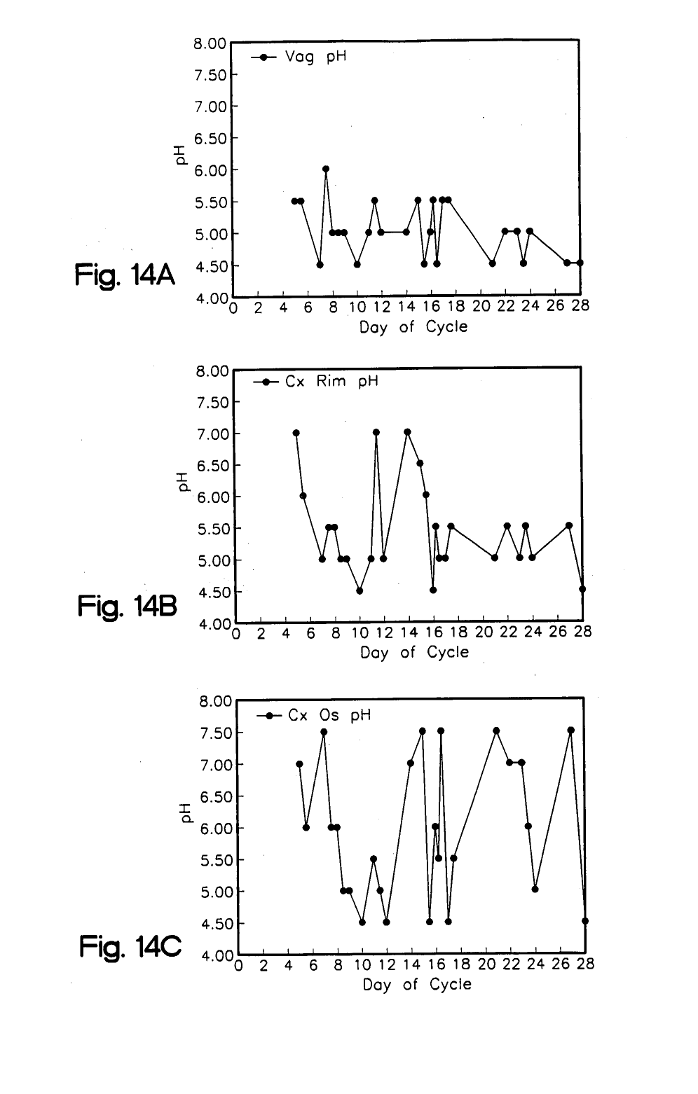

The vaginal and ectocervical pH for 26 women was determined using a vaginal pH probe in accordance with the present invention. Specifically, the pH level for each woman was measured along the vaginal wall, the rim of the ectocervix, and at the cervical os and plotted with respect to days since onset of menses (FIG. 14A-C).

FIG. 14A shows the pH levels measured along the walls of the vagina for the 26 women at various days of the menstrual cycle, excluding menstruation, varied from a low of about 4.50 to a high of about 6.0. This plot indicates that the pH measured along the vaginal wall of the 26 women was essentially the same (i.e., about 4.5 to about 6.0) over the menstrual cycle. Therefore prediction of ovulation based on this pH measurement alone could not be used accurately determine ovulation.

FIG. 14B shows the pH levels measured at the rim of the cervix for the 26 women at various days of the menstrual cycle, excluding menstruation, was high following menstruation (i.e., greater than about 6.0), lower after menstruation (i.e., about 4.5 to about 5.5), increased in women prior to and during onset of ovulation to a pH of about 7.0, and decreased to a pH of about 5.0 to 5.5 following ovulation and prior to menstruation. This plot indicates that the pH measured at rim of the cervix of the 26 women varied with increase in estrogen levels and ovulation could be predicted based on this pH measurement.

FIG. 14C shows the pH levels measured at the cervical os for the 26 women at various days of the menstrual cycle, excluding menstruation, was high following menstruation (i.e., greater than about 6.0), lower after menstruation (i.e., about 4.5 to about 5.5), increased in women prior to and during onset of ovulation to a pH of about 7.0, and remained high following ovulation and prior to menstruation. This indicates plot indicates that although the pH measured cervical os increased with estrogen level it remained high following ovulation and therefore cannot be relied on to predict ensuing ovulation or the end of ovulation.

1. A method of diagnosing ovulation in female mammal, the method comprising:
measuring the pH of the ectocervix of the female mammal; and
comparing the pH measured of the ectocervix to a reference value.

2. The method of claim 1, the reference value comprising a predetermined value based on previous pH measurements.

3. The method of claim 1, the reference value comprising a single normalized value or a range of normalized values.

4. The method of claim 1, further comprising measuring the pH along the vaginal wall and comparing the pH measured of the ectocervix with the pH measured along the vaginal wall.

5. The method of claim 4, the pH measured of the ectocervix and the pH measured along the vaginal wall being performed substantially concurrently.

6. The method of claim 1, further comprising measuring the pH at the endocervical canal and comparing the pH measured of ectocervix with the pH measured of the endocervical canal.

7. The method of claim 6, the pH measured of the ectocervix and the pH measured of the endocervical canal being performed substantially concurrently.

8. A method of diagnosing ovulation in female mammal, the method comprising:
measuring the pH of the ectocervix of the female mammal;
measuring the pH along the vaginal wall;
and comparing the pH measured of the ectocervix with the pH measured along the vaginal wall.

9. The method of claim 8, the pH measured of the ectocervix and the pH measured along the vaginal wall being performed substantially concurrently.

10. The method of claim 8, a substantially similar pH measured of the ectocervix and pH measured along the vaginal wall being indicative that ovulation in the female mammal has not occurred.

11. The method of claim of claim 8, a difference in pH measured of the ectocervix and the pH measured along the vaginal wall of at least about 1, being indicative that ovulation in the female mammal is imminent or has occurred.

12. An apparatus for diagnosing ovulation in a female mammal, the apparatus comprising:
an elongated member that extends along a central axis between a first end and a second end, the second end including an annular side wall sized to contact intimately and circumferentially an outer surface of a ectocervix of a female mammal, the annular side wall including a pH probe for measuring the pH of the ectocervix.

13. The apparatus of claim 12, the annular side wall being radially aligned from the central axis and comprising a pH sensitive material.

14. The apparatus of claim 13, the second end further including a tip that comprises a pH sensitive material, the tip extending from the second end and being sized to contact intimately an endocervical canal of the female.

15. The apparatus of claim 14, the elongated member being expandable from a first compressed size to a second expanded size.

16. The apparatus of claim 15, further comprising a sheath, the elongated member being capable of being received in the sheath when in a compressed size.

17. The apparatus of claim 16, the sheath preventing contact of the pH sensitive material of the annular side-wall during insertion of the member into a vagina of the female.

18. The apparatus of claim 12, the elongated member further including a lateral pH probe, the lateral probe being positioned on an outer surface of member between the first end and the second end.

19. The apparatus of claim 18, the lateral probe being capable of measuring the pH of a mid and/or lower portion of the vagina.

20. The apparatus of claim 12, the elongated member being substantially rigid housing.

21. The apparatus of claim 20, the pH probe comprising at least one of a pH electrode, thin-film pH sensors, or MEMS pH sensors.

22. The apparatus of claim 21, the member further comprising at least one of a temperature sensor, leuteinizing hormone (LH) sensor, or a pressure sensor.

23. The apparatus of claim 21, further comprising a means for receiving measured pH data and determining based on the measured pH data whether the female is ovulating.
